(19) United States
(12) Patent Application Publication
(10) Pub. No.: US 2013/0345585 A1
(43) Pub. Date: Dec. 26, 2013

(54) MONITORING APPARATUS FOR MONITORING A PHYSIOLOGICAL SIGNAL

(71) Applicants: Mathan Kumar Gopal Samy, Aachen (DE); Bin Yin, Shanghai (CN)

(72) Inventors: Mathan Kumar Gopal Samy, Aachen (DE); Bin Yin, Shanghai (CN)

(73) Assignee: KONINKLIJKE PHILIPS N.V., EINDHOVEN (NL)

(21) Appl. No.: 14/003,469

(22) PCT Filed: Feb. 8, 2012
(86) PCT No.: PCT/IB2012/050561

§ 371 Date: Sep. 6, 2013

(30) Foreign Application Priority Data
Mar. 11, 2011 (EP) 11157945.4

(51) Int. Cl. A61B 5/113; A61B 5/00; A61B 5/08

(52) U.S. Cl. CPC A61B 5/1135; A61B 5/08; A61B 5/7225; A61B 5/7203
USPC 600/529; 600/300; 600/595

(57) ABSTRACT

The invention relates to a monitoring apparatus for monitoring a physiological signal. A segmentation unit (4) determines signal segments from a physiological signal, which correspond to periods of the physiological signal, a classification unit (5) classifies the signal segments into a valid class and a non-valid class based on characteristics related to the signal segments, and a physiological information determination unit (7) determines physiological information from at least one of i)signal segments classified into the valid class and ii) signal segments classified into the non-valid class. The physiological information can therefore be determined based on the knowledge whether the respective signal segment is valid or not. For example, a physiological parameter like a breathing rate can be determined depending on valid adapted segments of the physiological signal, which are adapted to periods of the physiological signal. This improves the quality of determining physiological information from a physiological signal.

### FIELD OF THE INVENTION

The invention relates to a monitoring apparatus, a monitoring method and a monitoring computer program for monitoring a physiological signal.

### BACKGROUND OF THE INVENTION

U.S. Pat. No. 6,997,882 B1 discloses a method for monitoring respiratory functions of a subject. Acceleration signals are acquired from at least one accelerometer module attached to the subject. The acceleration signals are processed to obtain anterior-posterior acceleration signals representing anterior-posterior acceleration vectors largely free of medio-lateral acceleration vectors. An acceleration component that is due to breathing is extracted from the anterior-posterior acceleration signals, wherein the extraction comprises the application of a least means square adaptive noise-cancellation technique. The extracted acceleration component is likely to be adversely affected by non-breathing motion. The quality of the extracted acceleration component is therefore reduced.

### SUMMARY OF THE INVENTION

It is an object of the present invention to provide a monitoring apparatus, a monitoring method and a monitoring computer program for monitoring a physiological signal, wherein the quality of determining physiological information can be improved. In a first aspect of the present invention a monitoring apparatus for monitoring a physiological signal is presented, wherein the monitoring apparatus comprises:
a physiological signal providing unit for providing a periodic physiological signal,
a segmentation unit for determining signal segments from the physiological signal, which correspond to periods of the physiological signal,
a classification unit for classifying the signal segments into a valid class and a non-valid class based on characteristics related to the signal segments,
a physiological information determination unit for determining physiological information from at least one of i) signal segments classified into the valid class and ii) signal segments classified into the non-valid class.

Since the segmentation unit determines signal segments from the physiological signal, which correspond to periods of the physiological signal, wherein then the classification unit classifies the signal segments into a valid class and a non-valid class based on characteristics related to the signal segments, and the physiological information determination unit determines physiological information from at least one of i) signal segments classified into the valid class and ii) signal segments classified into the non-valid class, the physiological information can be determined based on the knowledge whether the respective signal segment is valid or not. For example, the physiological information determination unit can be adapted to determine a physiological parameter like a breathing rate depending on valid adapted segments of the physiological signal, which are adapted to periods of the physiological signal, for example, which are adapted to single breaths of a breathing signal. This allows improving the quality of determining physiological information from a periodic physiological signal.

The physiological signal providing unit can be a storing unit, in which the periodic physiological signal is stored already and from which the physiological signal can be retrieved for providing the same. The physiological signal providing unit can also be a receiving unit for receiving the periodic physiological signal via a wireless or wired data connection, wherein the physiological signal providing unit provides the received periodic physiological signal. The physiological signal providing unit can also be a measurement unit for measuring the periodic physiological signal. For example, the physiological signal providing unit can be an accelerometer for measuring an accelerometer breathing signal. The period of the physiological signal corresponds preferentially to single breath.

It is preferred that the segmentation unit is adapted to find valleys in the physiological signal and to determine a signal segment as a segment of the physiological signal between two neighbored valleys. The valleys can indicate a start and an end of a period of the physiological signal. Thus, by defining a signal segment by two neighbored valleys the signal segment can represent a period of the physiological signal.

It is further preferred that the segmentation unit is adapted to i) find valleys in the physiological signal, ii) apply a set of predefined rules to characteristics of the physiological signal around the found valleys, wherein the set of rules defines whether a found valley is a start or an end of a period of the physiological signal based on the characteristics of the physiological signal around the found valleys, iii) discard the found valleys which do not define a start or an end of a period, iv) determine a signal segment as a segment of the physiological signal between two neighbored non-discarded valleys. In a preferred embodiment, the segmentation unit is adapted to apply a set of rules according to which at least one of the amplitude, skewness and slope of the physiological signal before the respective found valley is compared with that of the respective physiological signal after the respective found valley, and to determine whether the respective found valley is caused by a beginning or an end of a period of the physiological signal depending on the comparison. The physiological signal can be corrupted by, for example, artifacts, noise and other imperfections, or may deviate from a sinusoidal waveform due to the nature of the respective measurement principle used for measuring the physiological signal, which may result in valleys which are not caused by a beginning or an end of a period of the physiological signal. Thus, by applying the set of rules and by discarding the found valleys which, according to the set of rules, do not define a start or an end of a period of the physiological signal, the quality of determining the signal segments can be improved.

The classification unit is adapted to classify the signal segments into a valid class and a non-valid class based on characteristics related to the signal segments. Characteristics related to the signal segments are, for example, temporal, spectral and spatial characteristics. They can be characteristics of the respective signal segment itself Characteristics related to the signal segments can also be characteristics which correspond to properties of a measuring unit used for measuring the physiological signal, the condition of the person or animal, et cetera, while the respective signal segment has been measured. For instance, the physiological signal can be an accelerometer signal, wherein the classification unit can be adapted to classify the signal segments based on a rotation angle defining the rotation of the accelerometer while the respective signal segment has been measured. The rotation angle is preferentially defined as the angle to which an accelerometer is rotated in space during a single period, i.e. the rotation angle can be defined as the difference between the orientation of the accelerometer at the start of the respective signal segment and the orientation at the end of the respective signal segment. For signal segments from motion artifacts the rotation angles are usually larger when compared to signal segments from physiological signals. In particular, breathing signals, which do not comprise motion artifacts, can in principle yield a zero net rotation angle during a single breathing period, i.e. the accelerometer returns substantially to its initial orientation at the end of one breath. The classification unit can also be adapted to classify the signal segments based on other characteristics related to the signal segments like the spectral entropy of the respective signal segment, the number of valleys within the respective signal segment and/or the shape of valleys within the respective signal segment.

Preferentially, the classification unit is adapted to use a decision tree classifier for classifying the signal segments into a valid class and a non-valid class. This allows classifying the signal segments in a simple way and in realtime.

It is also preferred that the classification unit is adapted to determine an accuracy value being indicative of the accuracy of classifying a signal segment into the valid class or the non-valid class depending on the respective signal segment. For example, if the classification unit is adapted to use a decision tree classifier for classifying the signal segments into a valid class and a non-valid class, a cross-validation of the decision tree classifier with ground truth can be performed, wherein an accuracy value can be determined for a certain leaf node, to which a valid label or a non-valid label has been assigned, by determining the ratio of correct decisions made at the leaf node to that of all decisions at the leaf node. The accuracy value can be used as an indication for the quality of the classification, which can be shown to a user and/or used in further processings.

In a preferred embodiment, the monitoring apparatus further comprises a classification correction unit for correcting the classification of the signal segments into the valid class and the non-valid class. In particular, the classification correction unit can be adapted to correct a classification of a certain signal segment, if the accuracy value of the certain signal segment is below a predefined accuracy threshold. For example, depending on the criticality of the physiological parameters and the application scenario, an appropriate value for the accuracy threshold can be set. In an embodiment, the classification correction unit can comprise assignments between a) physiological parameters and/or applications and b) accuracy thresholds, wherein the classification correction unit can use an accuracy threshold based on the assignments and the currently monitored physiological parameter and/or the current application. The classification correction unit can comprise a set of correction rules, which define, whether and how a classification of a segment has to be corrected based on at least one of correction features like the accuracy value, characteristics of the respective segment, characteristics of neighboring segments, user characteristics like a user activity level, environmental characteristics like the environmental temperature, the time of day, et cetera. For example, the set of rules can define that, if a segment is labeled as being valid and has an accuracy value being smaller than the accuracy threshold, which may be 60%, and if the temporarily neighboring segments are non-valid segments, the classification correction unit corrects the particular segment's label as non-valid. This allows improving the quality of the classification of the segments into valid and non-valid segments, thereby further improving the quality of the determination of the physiological parameter.

The physiological information determination unit can be adapted to determine a physiological parameter as the physiological information from the signal segments classified into the valid class. For example, a breathing rate or a heart rate can be determined as the physiological parameter. In particular, the physiological signal providing unit can be adapted to provide a breathing signal as the physiological signal, wherein the physiological information determination unit can be adapted to determine the respiration rate as the physiological parameter from the signal segments classified into the valid class by, for example, inverting the duration of a signal segment classified into the valid class. The physiological information determination unit can further be adapted to determine a physiological pattern as the physiological information from the signal segments classified into the valid class and from the signal segments classified into the non-valid class. The physiological pattern is, for example, a breathing pattern or a heart beat pattern, i.e. a cardiac pattern. In particular, the physiological information determination unit can be adapted to determine breathing patterns such as Cheyne-stokes respiration, periodic breathing, Apnea et cetera. For determining a breathing pattern, the physiological information determination unit is preferentially adapted to not only use valid segments, but also non-valid segments. In particular, the physiological information determination unit can be adapted to determine a breathing pattern based on valid and non-valid segments, which cover a duration of, for example, two minutes, and characteristics of these valid and non-valid segments. The physiological information determination unit can comprise a set of rules, which determine a breathing pattern based on these valid and non-valid segments and their characteristics. For example, the set of rules can define that, if a part of the physiological signal with contiguous non-valid segments with a variance being lower than a predefined variance threshold is followed by a part of the physiological signal with contiguous valid segments of modulated amplitudes, a Cheyne-stokes respiration pattern is present. The part of the physiological signal with contiguous non-valid segments with the low variance can represent apnea and the part of the physiological signal with contiguous valid segments of modulated amplitudes can represent hyperpnea.

It is preferred that the monitoring apparatus further comprises a pre-processing unit for pre-processing the physiological signal by performing at least one of the following: filtering, normalization, offset removing, down-sampling. The pre-processing can improve the quality of the physiological signal and/or reduce the computational load for further processing of the respiratory signal. For example, by down-sampling of the physiological signal, the computation time and load for further processing can be reduced. In an embodiment, the physiological signal is a respiratory signal and the respiratory signal is down-sampled to less than 20 Hz, preferentially to about 16 Hz.

In an embodiment, the physiological signal providing unit is adapted to provide several periodic physiological signals like a respiratory signal and a pulse signal. The segmentation unit can then be adapted to determine signal segments for the different physiological signals, the classification unit can be adapted to classify the signal segments of the different physiological signals, and the physiological information determination unit can be adapted to determine physiological parameters for the different physiological signals. For example, a respiration rate and a pulse rate can be determined. In order to use the physiological parameters determined for the different physiological signals for an evaluation of the condition of the monitored object, in particular, the condition of a human being or an animal, the pre-processing unit can be adapted to apply a phase correction to the different physiological signals such that they are in phase.

In an embodiment, the physiological signal is a breathing signal and the pre-processing unit is adapted to apply a band-pass filter of 0.1 to 2 Hz to the breathing signal. Thus, only frequencies pass the pre-processing unit, which may be correlated to the breathing, thereby improving the quality of the breathing signal.

In a further embodiment, the physiological signal providing unit is adapted to provide three physiological signals, which correspond to three axes of a tri-axial accelerometer, wherein the pre-processing unit is adapted to fuse the three physiological signals to a single physiological signal. For example, the pre-processing unit can apply a principle component analysis (PCA) to the physiological signal. The PCA transforms a data set of three correlated signals, which preferentially correspond to three different axes of an accelerometer, into a data set of three orthogonal signals, where the first principle component carries maximum variance. The pre-processing unit can be adapted to determine the first principle component of the PCA as the fused single physiological signal, thereby reducing three-dimensional data to one-dimensional data. This can lower the computational time and load for further processing.

Preferentially, the monitoring apparatus further comprises a display for displaying the determined physiological parameter.

In a further aspect of the present invention a monitoring method for monitoring a physiological signal is presented, wherein the monitoring method comprises:
providing a periodic physiological signal by a physiological signal providing unit,
determining signal segments from the physiological signal, which correspond to periods of the physiological signal, by a segmentation unit,
classifying the signal segments into a valid class and a non-valid class based on characteristics related to the signal segments by a classification unit,
determining physiological information from at least one of i) signal segments classified into the valid class and ii) signal segments classified into the non-valid class by a physiological information determination unit.

In a further aspect of the present invention a monitoring computer program for monitoring a physiological signal is presented, wherein the monitoring computer program comprises program code means for causing a monitoring apparatus as defined in claim 1 to carry out the steps of the monitoring method as defined in claim 14, when the computer program is run on a computer controlling the monitoring apparatus.

It shall be understood that the monitoring apparatus of claim 1, the monitoring method of claim 14 and the monitoring computer program of claim 15 have similar and/or identical preferred embodiments, in particular, as defined in the dependent claims.

It shall be understood that a preferred embodiment of the invention can also be any combination of the dependent claims with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of a monitoring apparatus for monitoring a physiological signal,

FIGS. 2 and 3 show exemplarily a part of a breathing signal with valleys and peaks,

FIG. 4 shows exemplarily a breathing signal with valid signal segments and non-valid signal segments, and

FIG. 5 shows a flowchart exemplarily illustrating an embodiment of a monitoring method for monitoring a physiological signal.

### DETAILED DESCRIPTION OF EMBODIMENTS

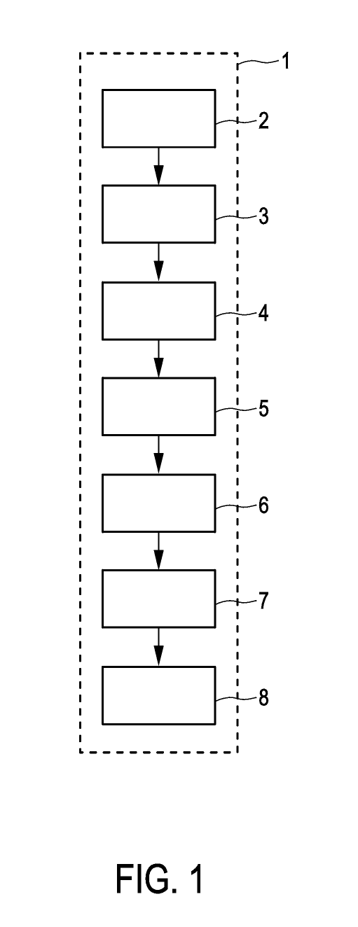

FIG. 1 shows schematically and exemplarily an embodiment of a monitoring apparatus for monitoring a physiological signal. The monitoring apparatus 1 comprises a physiological signal providing unit 2 for providing a periodic physiological signal. In this embodiment, the physiological signal providing unit 2 is a storing unit, in which the periodic physiological signal is stored already. The physiological signal is preferentially an accelerometer breathing signal, which has been measured by using an accelerometer. The accelerometer is an electromechanical sensor that measures both, gravitational, induced by the earth gravity (g=9.8 ms²), and inertia accelerations induced by movements. In particular, the accelerometer is a three-axial accelerometer, which is attached to a thorax, preferably on the left rib cage, of a human being, in order to measure projections of the gravity vector onto its sensing axes. During breathing, the movement of the rib cage rotates the sensor in an earth reference system, thus causing a change of the projections of the gravity vector on the three axes, which is reflected on the sensor axes signals. Thus, the accelerometer is preferentially used as an inclinometer, in order to measure accelerometer breathing signals. By using the accelerometer, a cable-less, unobtrusive, low-cost and continuous respiration monitoring can be provided. Instead of or in addition to attaching the accelerometer to the thorax, the accelerometer can also be attached to another part of a human being, which moves with breathing, like the abdomen.

In another embodiment, the physiological signal providing unit is adapted to measure the periodic physiological signal. In particular, the physiological signal providing unit can comprise an accelerometer for measuring an accelerometer breathing signal, an analogue-to-digital converter for converting the measured analogue accelerometer breathing signal to a digital accelerometer breathing signal and a buffer for storing the digital accelerometer breathing signal before providing it to a pre-processing unit 3.

The pre-processing unit 3 is adapted to pre-process the provided physiological signal by performing at least one of the following: filtering, normalization, offset-removing and down-sampling. This pre-processing is preferentially performed such that the following signal processing is simplified, without losing relevant information. For example, the pre-processing unit 3 can be adapted to apply a band-pass filter of 0.1-1 Hz or 0.1-2 Hz to the breathing signal. Thus, the filtering can be performed such that only frequencies pass the pre-processing unit 3, which may be correlated to the breathing, thereby improving the quality of the breathing signal. The band-pass filter of 0.1-1 Hz corresponds to the normal breathing rate of 6-60 breaths per minute. The band-pass filter of 0.1-2 Hz also considers neonatal breathing.

In this embodiment, the physiological signal providing unit 2 is adapted to provide three accelerometer signals of a tri-axial accelerometer as breathing signals. The three accelerometer signals, which correspond to the three axes of the accelerometer, are preferentially fused into one breathing signal by using PCA by the pre-processing unit 3. The first principle component of the PCA is preferentially the single breathing signal, which is used for further processing.

The monitoring apparatus 1 further comprises a segmentation unit 4 for determining signal segments from the physiological signal, i.e., in this embodiment, the breathing signal, which correspond to periods of the physiological signal. The segments can be regarded as breath candidates, wherein the segmentation unit 4 prepares the breathing signal for breath detection by generating the breath candidates, which are basic units of a classification, which will be described further below. A typical breathing cycle resembles a sinusoidal-shaped breathing signal with a peak separating inspiration and expiration, while the start of inspiration and the end of expiration are marked by local minima in the breathing signal.

The segmentation unit 4 can be adapted to find valleys in the physiological signal and to determine a signal segment as a segment of the physiological signal between two neighbored valleys. Thus, the segmentation unit 4 can be adapted to find valleys in the breathing signal, which may be defined as local minima below the mean of the breathing signal, to identify start and end of a breath candidate. Since not all of the valleys may be a true beginning/or end of a breath due to, for example, the nature of the measurement principle used for measuring the physiological signal, small artifacts, noise and other imperfections, invalid valleys may be present, which may lead to false candidates. The segmentation unit 4 can therefore be adapted to apply a set of predefined rules to characteristics of the breathing signal around the found valleys, wherein the set of rules defines, whether a found valley is a start of an end of a period of the breathing signal, based on the characteristics of the breathing signal around the found valleys. In particular, the segmentation unit 4 can be adapted to apply a set of rules according to which at least one of the amplitude, the skewness and slope of the breathing signal before the respective found valley is compared with that of the respective breathing signal after the respective found valley, wherein it is determined that the respective found valley is caused by a beginning or an end of a period of the breathing signal depending on the comparison. The set of rules can be determined by calibration or training, wherein the set of rules is defined such that for the calibration or training breathing signals the accuracy of determining, whether the respective found valley is caused by a beginning or an end of a period of the breathing signal or not, is optimized. Thus, a set of rules can be defined by learning from occurrences of, for example, false candidates, wherein the set of rules are defined such that the occurrences of false candidates are minimized.

The segmentation unit 4 can be further adapted to discard the found valleys, which do not define a start or an end of a period of the breathing signal and to determine a signal segment as a segment of the breathing signal between two neighboring non-discarded valleys.

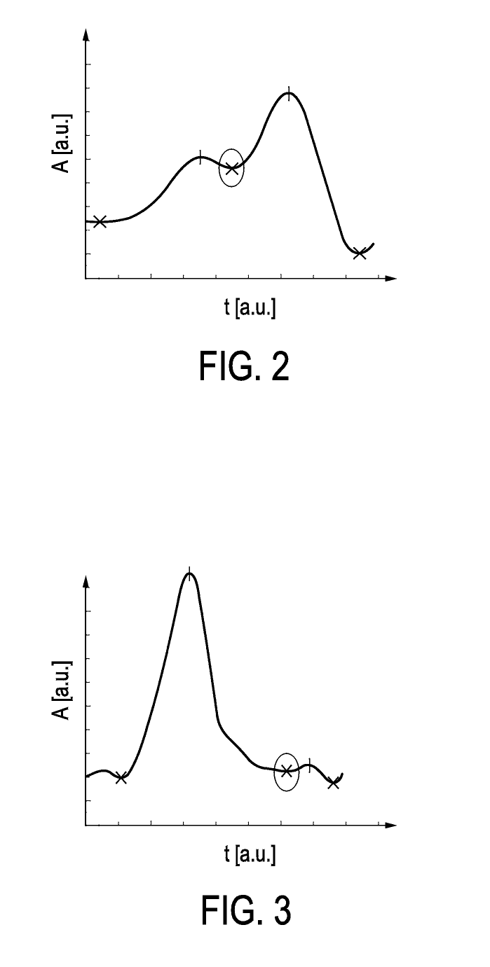

FIGS. 2 and 3 show schematically and exemplarily a part of a fused accelerometer breathing signal, in particular, the amplitude A of the signal in arbitrary units depending on the time t in arbitrary units. In these figures, the crosses indicate valleys, the vertical lines indicate peaks and the ellipses indicate valleys to be removed. The segmentation unit 4 can be adapted to find the valleys, which are denoted in FIGS. 2 and 3 by ellipses, in order to identify these valleys as valleys, which do not define a start or an end of a breathing period.

The monitoring apparatus further comprises a classification unit 5 for classifying the signal segments into a valid class and a non-valid class based on characteristics related to the signal segments. Thus, the classification unit 5 is preferentially adapted to calculate characteristics, which can also be regarded as a feature, related to the respective signal element, i.e. to the respective breath candidate, and to classify the respective signal segment depending on the calculated characteristics. The characteristics can be, for example, at least one of temporal, spectral and spatial characteristics related to the respective signal element. For example, a rotation angle defining the rotation of the accelerometer while the respective signal segment has been measured can be calculated as a spatial characteristic, the spectral entropy of the respective signal segment can be determined as a spectral characteristic, and the number of valleys of the respective signal segment and the shape of valleys of the respective signal segment can be calculated as temporal characteristics. The rotation angle is preferentially defined as the angle to which an accelerometer is rotated in space during a single period, i.e. the rotation angle can be defined as the difference between the orientation of the accelerometer at the start of the respective signal segment and the orientation at the end of the respective signal segment. The classification unit 5 can, for example, be adapted to classify a respective signal segment as valid, if the rotation angle is smaller than a predefined rotation angle threshold, and to classify the respective signal segment as non-valid, if the rotation angle is larger than the predefined rotation angle threshold. The rotation angle threshold can be predefined by calibration or training with signal segments, which are unknown to be valid or non-valid.

The classification unit 5 is preferentially adapted to determine a feature vector, which represents the characteristics related to the respective signal element, for the signal segments, i.e. for the breath candidates. The classification unit then preferentially uses the feature vector to categorize a particular breath candidate as either valid or non-valid, i.e. as a breath segment or a non-breath segment. The classification unit 5 can use various types of classifiers that may fulfill the task. Preferentially, the classification unit 5 uses a decision tree classifier, which allows to classify the signal segments in a simple way and in realtime.

The decision tree classifier is also preferentially trained or calibrated by using signal segments, of which it is known, whether they are valid or non-valid. For example, the decision tree classifier can be trained from manually annotated breathing data, wherein it is annotated whether the respective signal segment is valid or non-valid. The breathing data can be breathing data of several persons located in one or several hospitals.

In an embodiment, the feature vector comprises several features, spanning across in time, frequency and/or spatial domains. For example, the feature vector can comprise at least one of the following features: width, mean, median, amplitude variance, peak amplitude, amplitude at signal element start, amplitude at signal element end, relative location of peak within the respective signal element, positive width, number of valleys inside signal segment but above mean, slope of line passing through signal segment start and end amplitudes, area under the signal segment curve, dominant frequency, magnitude at dominant frequency, spectral entropy, spectral centroid, magnitude in several, in particular, four, different predefined frequency bands, rotation angle et cetera. The several frequency bands can be predefined by using training data sets comprising signal segments of which the correct assignment to the valid class or the non-valid class is known, wherein the frequency bands are chosen such that the number of incorrectly classified signal segments of the training data set is minimized.

The classification unit 5 can be adapted to pre-classify the signal segments before performing the above described classification. Preferentially, the pre-classification is performed by using a decision tree pre-classifier, which is smaller than the decision tree classifier described above. In particular, less features are determined for a signal segment and the pre-classification is performed based on these few features. For example, the features used for the pre-classification can be easy-to-compute features like the width or amplitude variance of the respective signal segment.

The pre-classification is preferentially used to identify obvious non-valid signal segments and can be trained be using a training data set. The decision tree pre-classifier can, for example, be adapted such that a signal segment having an amplitude variance smaller than an apnea threshold is regarded as being an obvious non-valid segment, because it is assumed that such a signal segment is inside apnea. Moreover, the pre-classification can be performed such that a signal segment having an amplitude variance above a motion threshold is identified as a non-valid signal segment, because it is assumed that the relatively high variance of the amplitude is caused by a motion artifact. The main classification is then performed on the signal segments, which have not been identified as being obvious non-valid signal segments by the pre-classification. The main classification is therefore performed on less signal segments, thereby reducing the computational load of performing the main classification.

The decision tree classifier performing the main classification and the optional decision tree pre-classifier performing the pre-classification can be, for example, a standard C 4.5 decision tree classifier or a classification and regression tree (CART).

The classification unit 5 is preferentially further adapted to determine an accuracy value being indicative of the accuracy of classifying a signal segment into the valid class or the non-valid class depending on the respective signal segment. This accuracy value, which is indicative of the accuracy of classifying the respective signal segment, i.e., which is indicative of the label decision, is directly correlated to the quality of the corresponding breath. The classification unit 5 can be adapted to cross-validate the decision tree classifier with ground truth and to determine the accuracy value as a ratio of correct decisions made at the leaf, which assigns a valid or non-valid label to the respective signal segment, to that of all decisions at the leaf. In an embodiment, a training data set comprising signal segments, of which is it known whether they are valid or non-valid signal segments, is classified by the decision tree classifier. Since the correct classification of the respective signal segment is known, the classification resulting by using the decision tree classifier can be compared with the correct classification, in order to determine for each leaf of the decision tree classifier an accuracy value. For example, the accuracy value can be defined as the number of correct decisions made at a respective leaf divided by the overall number of decisions made at the respective leaf After for each leaf an accuracy value has been determined, the decision tree classifier can be applied to an actual signal segment, of which the correct classification is not known, wherein, after the classification has been performed, the respective actual signal segment is assigned to a certain leaf of the decision tree classifier, thereby defining the class of the respective actual signal segment and the corresponding accuracy value.

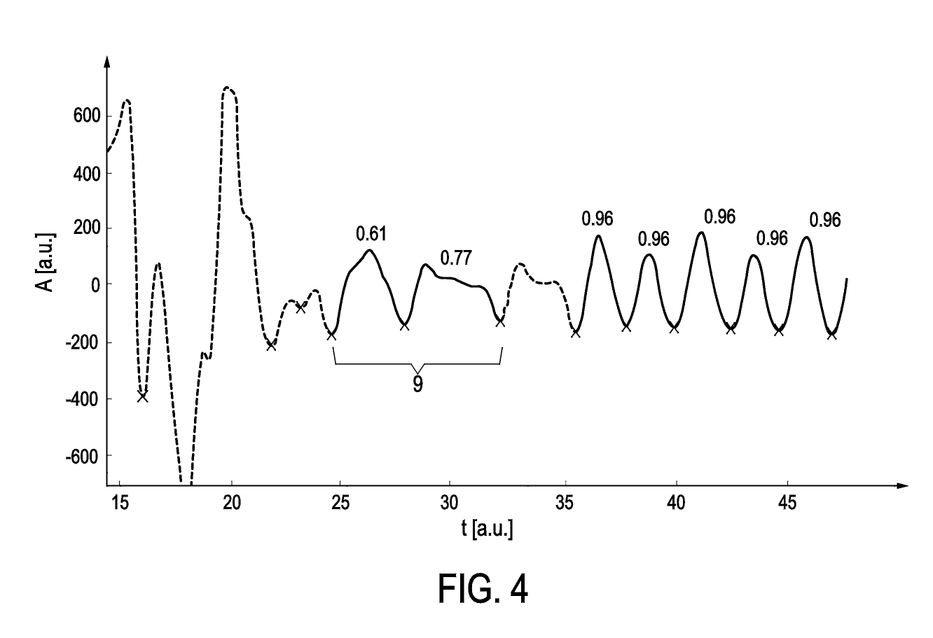

FIG. 4 shows schematically and exemplarily the amplitude A of a breathing signal depending on the time t. The different signal segments are defined by the valleys, which are indicated by crosses. The valid signal segments are denoted by a solid line and the non-valid signal segments are denoted by a dashed line. The numbers shown in FIG. 4 above the respective signal segment indicate the respective accuracy value.

As can be seen in FIG. 4, the signal segments 9 deviate from a sinusoidal shape and have a lower accuracy value. This deviation and the small accuracy values may be caused by an influence of the accelerometer signal by non-breathing motion.

The monitoring apparatus 1 preferentially further comprises a classification correction unit 6 for correcting the classification of the signal segments into the valid class and the non-valid class. In particular, depending on the accuracy in labeling and the knowledge of surrounding labels, a classifier decision can be revoked to improve reliability.

The classification correction unit 6 can be adapted to correct a classification of a certain signal segment, if the accuracy value of the certain signal segment is below a predefined accuracy threshold. For example, depending on the criticallity of the physiological parameters and the application scenario, an appropriate value for the accuracy threshold can be set. In an embodiment, the classification correction unit 6 can comprise assignments between a) physiological parameters and/or applications and b) accuracy thresholds, wherein the classification correction unit 6 can use an accuracy threshold based on the assignments and the currently monitored physiological parameter and/or the current application. For instance, the accuracy thresholds can be predefined such that the classification of the signal segments 9 shown in FIG. 4 and having an accuracy value smaller than 0.8 is corrected from the valid class to the non-valid class.

The classification correction unit 6 can comprise a set of correction rules, which define, whether and how a classification of a segment has to be corrected based on the accuracy threshold, characteristics of the respective segment, characteristics of neighboring segments, user characteristics like a user activity level, environmental characteristics like the environmental temperature, the time of day, et cetera. For example, the set of rules can define that, if a segment is labeled as being valid and has an accuracy value being smaller than the accuracy threshold, which may be 60%, and if the temporarily neighboring segments are non-valid segments, the classification correction unit 6 corrects the particular segment's label as non-valid. This allows improving the quality of the classification of the segments into valid and non-valid segments, thereby further improving the quality of the determination of the physiological parameter.

The monitoring apparatus 1 further comprises a physiological information determination unit 7 for determining physiological information from at least one of i) signal segments classified into the valid class and ii) signal segments classified into the non-valid class. In particular, the physiological information determination unit 7 is adapted to determine a physiological parameter from the signal segments classified into the valid class. In this embodiment, the physiological information determination unit 7 is adapted to determine the respiration rate as the physiological parameter from the signal segments classified into the valid class. For example, the physiological information determination unit 7 can be adapted to determine the respiration rate by inverting the duration of a signal segment classified into the valid class. The physiological information determination unit 7 can further be adapted to determine breathing patterns such as Cheyne-stokes respiration, periodic breathing, apnea, et cetera. For determining a breathing pattern, the physiological information determination unit 7 is preferentially adapted to not only use valid segments, but also non-valid segments. In particular, the physiological information determination unit 7 can be adapted to determine a breathing pattern based on the valid and non-valid labels of signal segments, which cover a duration of, for example, two minutes, and characteristics of these valid and non-valid segments. The physiological information determination unit 7 can comprise a set of rules, which determine a breathing pattern based on the valid and non-valid labels of the signal segments and the characteristics of the signal segments. For example, the set of rules can define that, if a part of the physiological signal with contiguous non-valid segments, which have preferentially a duration of at least 10 seconds, with a variance being lower than a predefined variance threshold is followed by a part of the physiological signal with contiguous valid segments of modulated amplitudes, a Cheyne-stokes respiration pattern is present. The part of the physiological signal with contiguous non-valid segments with the low variance can represent apnea and the part of the physiological signal with contiguous valid segments of modulated amplitudes can represent hyperpnea. Also the set of rules used by the physiological information determination unit 7 can be defined by calibration or training, wherein signal segments representing a known physiological parameter and/or a known physiological pattern are used.

The monitoring apparatus 1 further comprises a display 8 for displaying, for example, the physiological parameter like the breathing rate, the physiological pattern like the breathing pattern, the valid signals and/or the non-valid signals. In particular, the display can be adapted to show the valid and non-valid signal segments as exemplarily shown in FIG. 4.

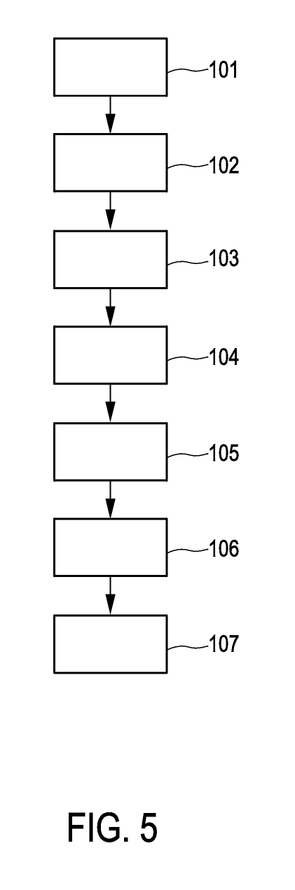

In the following an embodiment of a monitoring method for monitoring a physiological signal will exemplarily be described with reference to a flowchart shown in FIG. 5.

In step 101, the physiological signal providing unit 2 provides periodic physiological signals, which are, in this embodiment, accelerometer breathing signals. In step 102, the physiological signals are pre-processed by the pre-processing unit 3. In particular, the physiological signals are filtered by using a band-pass filter of 0.1-2 Hz. Moreover, the pre-processing unit 3 fuses the accelerometer breathing signals, which are preferentially three accelerometer breathing signals of a three-axial accelerometer, to a single breathing signal by PCA. In step 103, the segmentation unit 4 determines signal segments from the pre-processed physiological signal, which correspond to periods of the physiological signal, and, in step 104, the classification unit 5 classifies the signal segments into a valid class and a non-valid class based on characteristics related to the signal segments. In step 105, the classification correction unit 6 corrects the classification of the signal segments into the valid class and the non-valid class, if required, and, in step 106, the physiological information determination unit 7 determines physiological information, for example, a physiological parameter like the breathing rate from the signal segments classified into the valid class or a physiological pattern like the breathing pattern from the signal segments classified into the valid class and the signal segments classified into the non-valid class. In step 107, at least the determined physiological parameter and/or the physiological pattern is shown on the display 8.

Although in the above described embodiments, the physiological signal is an accelerometer breathing signal, the monitoring apparatus and the monitoring method can also be adapted to monitor a breathing signal, which is measured by another device like a respiratory belt. The monitoring apparatus and the monitoring method can also be adapted to monitor another physiological signal like a cardiac signal, in particular, like an electro-cardiography signal.

The monitoring apparatus and the monitoring method can be adapted to monitor one or several physiological signals. For example, one or several accelerometers can be used for monitoring one or several breathing signals. Moreover, the monitoring apparatus and the monitoring method can be adapted to monitor different physiological signals like a breathing signal and a cardiac signal, simultaneously.

Physiological parameters like breathing and heart rates can have a high clinical value as an early indicator of patient's health deterioration. Traditionally, patients in the intensive and medium care units are well-monitored, while those in medical and post-surgical care units are not. Generally, a continuous monitoring of patient's physiological parameters over a long time period is an arduous task due to the shortage of nursing personnel, lack of supervision and unavailability of a suitable monitoring system, which may lead to a sub-optimal care. The above described monitoring apparatus can be a reliable, automatic and easy-to-use device to measure physiological parameters for the general ward patients.

The acceleration sensor is preferentially attached to the patient's thorax or abdomen and can be used to detect breathing effort along with information such as heart-beating, body-posture and activity level of the patient. The acceleration sensor can be attached to patients, who are physically more active than traditionally monitored patients of an intensive care unit (ICU), wherein the sensor signals can be mixed-up with accelerations due to patient's body movements. The monitoring apparatus and the monitoring method can be adapted to ensure a reliable interpretation of the measured data by deriving the physiological parameters from clinical relevant signals rather than motion-contaminated signals. By doing so, false alarms are limited and the patient's status can be properly supervised. In particular, the monitoring apparatus and the monitoring method are preferentially adapted to discard motion-contaminated signals, i.e. motion-contaminated signal segments, which are classified as non-valid, and reliably extract respiration/pulse rates and trends from the rest of the signal. Thus, an automatic algorithm can be provided, which intelligently identifies and removes motion-contaminated measurements to make a continuous monitoring of vital body signs in general ward patients meaningful.

The monitoring apparatus and monitoring method are preferentially adapted to accurately detect every single breath in a breathing signal. Thus, in contrast to a fixed time frame analysis, in which a breathing signal is segmented and processed in fixed-sized frames of, for example, 25 s, the monitoring apparatus and monitoring method can be adapted to operate at the finest resolution and thus captures every single breath that is valid, thereby increasing the availability of breathing information. The output such as the respiration rate (RR), the accuracy value, which can also be regarded as a confidence index (CI), the breathing pattern, et cetera, are preferentially sent to the display to notify an observer. The monitoring apparatus and monitoring method detect preferentially every single breath after an adaptive segmentation of the breathing signal. A sequence of operations is performed on the breathing signal to calculate, for example, RR and a corresponding CI. The CI quantifies preferentially the confidence in evaluation of the respiratory rate from a single breath. It reflects the accuracy in the breath detection and the quality of the breath.

The breathing signal is preferentially a digitized signal of an accelerometer and is preferentially buffered up to one cycle of respiration, for example, for 10 seconds, before being pre-processed by the pre-processing unit which preferentially carries out operations such as filtering, DC-removal, normalization, et cetera on the digitized and buffered raw sensor signals. The segmentation unit then preferentially demarcates the signal to generate breath candidates based on pre-defined rules. In comparison to a fixed-frame classification, the breath candidates, i.e. the signal segments, can be seen as the high-resolution frames with time-varying lengths, which are the basic units, on which the classification unit operates. The classification unit calculates preferentially a set of breath-specific features for each breath-candidate, which are then classified into “breathing”, i.e. into valid, and “non-breathing”, i.e. non-valid, categories using a classification algorithm such as a decision tree. The physiological information determination unit then preferentially calculates the respiratory rate along with the corresponding index of confidence from the breath candidates that were classified as “breathing”.

The monitoring apparatus and the monitoring method can be adapted to be applied in a setting, where vital body signs such as respiration are monitored by using one or more sensors, in particular, one or more accelerometers. The monitoring apparatus and the monitoring method can be adapted to monitor respiration in general wards at the hospital. But, the monitoring apparatus and the monitoring method can also be adapted for ICU monitoring and home healthcare.

Although in above described embodiments PCA is used as a technique for fusing physiological signals, also other fusion techniques can be used like weighted sum beamforming (WSB), geometrical coordinates rotation and other heuristic fusion methods.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Calculations like the determination of signal segments, the classification of the signal segments or the determination of physiological information, performed by one or several units or devices, can be performed by any other number of units or devices. For example, steps 102 to 106 can be performed by a single unit or by any other number of different units. The calculations and/or the control of the monitoring apparatus in accordance with the monitoring method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a monitoring apparatus for monitoring a physiological signal. A segmentation unit determines signal segments from a physiological signal, which correspond to periods of the physiological signal, a classification unit classifies the signal segments into a valid class and a non-valid class based on characteristics related to the signal segments, and a physiological information determination unit determines physiological information from at least one of i) signal segments classified into the valid class and ii) signal segments classified into the non-valid class. The physiological information can therefore be determined based on the knowledge whether the respective signal segment is valid or not. For example, a physiological parameter like a breathing rate can be determined depending on valid adapted segments of the physiological signal, which are adapted to periods of the physiological signal. This improves the quality of determining physiological information from a physiological signal.

1. A monitoring apparatus for monitoring a physiological signal, the monitoring apparatus comprising:
a physiological signal providing unit for providing a periodic physiological signal,
a segmentation unit for determining signal segments from the physiological signal, which correspond to periods of the physiological signal,
a classification unit for classifying the signal segments into a valid class and a non-valid class based on characteristics related to the signal segments,
a physiological information determination unit for determining physiological information from at least one of i) signal segments classified into the valid class and ii) signal segments classified into the non-valid class.

2. The monitoring apparatus as defined in claim 1, wherein the segmentation unit is adapted to:
find valleys in the physiological signal,
determine a signal segment as a segment of the physiological signal between two neighbored valleys.

3. The monitoring apparatus as defined in claim 1, wherein the segmentation unit is adapted to:
find valleys in the physiological signal,
apply a set of predefined rules to characteristics of the physiological signal around the found valleys, wherein the set of rules defines whether a found valley is a start or an end of a period of the physiological signal based on the characteristics of the physiological signal around the found valleys,
discard the found valleys which do not define a start or an end of a period,
determine a signal segment as a segment of the physiological signal between two neighbored non-discarded valleys.

4. The monitoring apparatus as defined in claim 3, wherein the segmentation unit is adapted to:
apply a set of rules according to which at least one of the amplitude, skewness and slope of the physiological signal before the respective found valley is compared with that of the respective physiological signal after the respective found valley,
determine whether the respective found valley is caused by a beginning or an end of a period of the physiological signal depending on the comparison.

5. The monitoring apparatus as defined in claim 1, wherein the classification unit is adapted to classify the signal segments based on at least one of the temporal, the spectral and the spatial characteristics of the respective signal segment.

6. The monitoring apparatus as defined in claim 1, wherein the physiological signal is an accelerometer signal measured by an accelerometer and wherein the classification unit is adapted to classify the signal segments based on a rotation angle defining a rotation of the accelerometer while the respective signal segment has been measured.

7. The monitoring apparatus as defined in claim 1, wherein the classification unit is adapted to use a decision tree classifier for classifying the signal segments into a valid class and a non-valid class.

8. The monitoring apparatus as defined in claim 1, wherein the classification unit further adapted to determine an accuracy value being indicative of the accuracy of classifying a signal segment into the valid class or the non-valid class depending on the respective signal segment.

9. The monitoring apparatus as defined in claim 1, wherein the monitoring apparatus further comprises a classification correction unit for correcting the classification of the signal segments into the valid class and the non-valid class.

10. The monitoring apparatus as defined in claim 1, wherein the physiological information determination unit adapted to determine at least one of i) a physiological parameter as the physiological information from the signal segments classified into the valid class and ii) a physiological pattern as the physiological information from the signal segments classified into the valid class and from the signal segments classified into the non-valid class.

11. The monitoring apparatus as defined in claim 10, wherein the physiological signal providing unit is adapted to provide a breathing signal as the physiological signal and wherein the physiological information determination unit is adapted to determine the respiration rate as the physiological parameter from the signal segments classified into the valid class.

12. The monitoring apparatus as defined in claim 1, wherein the monitoring apparatus further comprises a pre-processing unit for pre-processing the physiological signal by performing at least one of the following: filtering, normalization, offset removing, down-sampling.

13. The monitoring apparatus as defined in claim 12, wherein the physiological signal providing unit is adapted to provide three physiological signals, which correspond to three axes of a tri-axial accelerometer, wherein the pre-processing unit is adapted to fuse the three physiological signals to a single physiological signal.

14. A monitoring method for monitoring a physiological signal, the monitoring method comprising:
providing a periodic physiological signal by a physiological signal providing unit,
determining signal segments from the physiological signal, which correspond to periods of the physiological signal, by a segmentation unit,
classifying the signal segments into a valid class and a non-valid class based on characteristics related to the signal segments by a classification unit,
determining physiological information from at least one of i) signal segments classified into the valid class and ii) signal segments classified into the non-valid class by a physiological information determination unit.

15. A monitoring computer program for monitoring a physiological signal, the monitoring computer program comprising program code means for causing a monitoring apparatus as defined in claim 1 to carry out the steps of the monitoring method, when the computer program is rim on a computer controlling the monitoring apparatus.
